(19) United States
(12) Patent Application Publication
(10) Pub. No.: US 2009/0192820 A1
(43) Pub. Date: Jul. 30, 2009

(54) ASSAY SYSTEM AND METHOD

(75) Inventors: Maarten Peter Bodlaender, Eindhoven (NL); Wilhelmus Johannes Joseph Stut, Eindhoven (NL); Erik Petrus Nicolaas Damen, Doorwerth (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/299,776

(22) PCT Filed: May 2, 2007
(86) PCT No.: PCT/IB2007/051619

§ 371 Date: Nov. 6, 2008

(30) Foreign Application Priority Data
May 9, 2006 (EP) 06113708.9

(51) Int. Cl. G06Q 50/00; G01N 33/48

(52) U.S. Cl. 705/2; 422/68.1

(57) ABSTRACT

Assay system, comprising: at least one first assay device (IA) that is configured to assay one or more samples and to provide at least one assay result; and at least one distribution system (10) which is configured to distribute at least one second assay device (IB) to a user of the at least first assay device (IC). The invention also relates to an assay method.

### FIELD OF THE INVENTION

The invention relates to an assay system comprising at least one first assay device that is configured to assay one or more samples and to provide at least one assay result.

The invention also relates to an assay method.

### BACKGROUND OF THE INVENTION

International patent application WO 95/33996 of Zwanziger et al. discloses a home test kit for use in testing for a disease or a physiological condition, with telephone verification of test results. The known home test kit facilitates the delivery of any necessary counseling as a result of the outcome of a test. During use, an individual can obtain a sample of physiological fluid from him or herself. The sample can be introduced into an assay device to produce a coded pattern indicative of the presence or absence of the disease. The individual can transmit the coded pattern to a remote location for interpretation, for example by telephone. Then, the individual can receive, from the remote location, an interpretation of the coded pattern together with any counseling which may be appropriate in view of the interpretation of the coded pattern. In this way, the remote location has to be used for interpretation of the test.

### SUMMARY OF THE INVENTION

The present invention aims to provide an improved assay system and assay method.

In an embodiment of the invention, the assay system is characterized by at least one distribution system, which is configured to distribute at least one second assay device to a user of a first assay device.

In this way, a relatively efficient assay system can be provided, suitable, for example, to screen a large group of people (for example: the population of a country, clients of an insurance company, or different groups) in a reliable and economical manner. Besides, the system can simply be configured to automatically adapt to a user situation and/or user motivation, to provide more reliable screening results.

In a further embodiment, the system can comprise at least one receiving system which is configured to receive assay data or information, the assay data or information relating to, being based on and/or comprising one or more assay results of the assay devices and/or comprising information that the assaying has failed. In the latter case, advantageously, the receiving system can be configured to receive at least parts of distributed assay devices and to perform at least one of the following: detect damage and/or malfunction of received assay devices or of parts thereof, read data or information from received assay devices or parts thereof, recycle received assay devices or parts thereof.

In a further embodiment, the assay system can comprise:
at least a third assay device, the third assay device being configured differently with respect to the configuration of the first assay device, and being configured differently with respect to the configuration of the second assay device, the at least one distribution system being configured to distribute at least one third assay device to a user of the at least first and/or second assay device.

For example, in an embodiment, the second assay device can have a higher assay sensitivity than the first assay device, and the third assay device can be more robust than the first assay device.

Moreover, according to an embodiment, the distribution system can be configured to randomly select users by randomly selecting user names and/or user addresses from at least one database containing such names and/or addresses, and can be configured to automatically notify selected users to fetch or receive a first assay device and/or to distribute first assay devices to randomly selected users.

Also, for example, in an embodiment, the distribution system can be configured to select users based on one or more medical selection criteria, and can be configured to automatically notify selected users to fetch or receive a first assay device and/or to distribute first assay devices to selected users.

Besides, as an example, the distribution system can be configured to select all users by selecting all user names and/or user addresses from at least one database containing such names and/or addresses, and can be configured to automatically notify all selected users to fetch or receive first assay devices and/or to distribute first assay devices to all users. According to an embodiment, at least one of the assay devices can include a lottery ticket. In an advantageous embodiment, the at least one distribution system comprises at least one distributor configured to hold a plurality of first assay devices, the distributor being located in or near one of the following: a shopping area, an entertainment area, a public transport area, a hospital.

Also, for example, the assay system can be configured to provide a user of a first assay device, in case the user has used the assay device to assay one or more samples, with at least one of the following: a gift, a price, a lottery ticket, a fee reduction, a reduction on an insurance fee. Then, in a further embodiment, the at least one first assay device can be configured to provide an assay result, which result also determines the gift, price, lottery number of the lottery ticket, fee reduction, or the reduction on the insurance fee.

Also, in an embodiment of the invention, there is provided an assay method, comprising:
distributing at least one first assay device to be used by a user to assay one or more samples; and
providing at least a second assay device;
distributing the second assay device to a user of a first assay device.

This method can utilize an above-mentioned assay system according to the invention, and can provide above-mentioned advantages concerning assaying large groups of individuals.

Besides, an embodiment of the invention provides a method to manage an insurance system, comprising utilizing a system according to the invention and/or a method according to the invention to assay users of the insurance system.

For example, in a further embodiment, the insurance system can be configured to provide a reduction on an insurance fee to users who have used at least one of the assay devices.

Also, an embodiment of the invention provides a computer program comprising computer-readable instructions and being configured to carry out a method according to the invention when run by a computer, and also provides an information carrier comprising a computer program.

### BRIEF DESCRIPTION OF THE DRAWINGS

Further advantageous embodiments of the invention are described in the dependent claims. These and other aspects of the invention will be apparent from and elucidated with reference to the embodiments described hereafter.

FIG. 1 schematically depicts a top view of a first embodiment of an assay device;

FIG. 2 schematically depicts a top view of a second embodiment of an assay device;

FIG. 3 schematically depicts a top view of a third embodiment of an assay device;

FIG. 4 schematically depicts an assay system;

FIG. 5 depicts a flow chart of an assay method which utilizes the assay system shown in FIG. 4;

FIG. 6 shows an embodiment of the invention, during use; and

FIG. 7 shows a flow chart of an alternative assay method.

### DETAILED DESCRIPTION

In the present application, similar or corresponding features are indicated by similar or corresponding reference signs.

There are 20.8 million people in the United States, or 7% of the population, who have diabetes. While an estimated 14.6 million have been diagnosed with diabetes, 6.2 million people (or nearly one-third) are unaware that they have the disease.

Today, in order to determine whether or not a person has pre-diabetes or diabetes, health care providers conduct a Fasting Plasma Glucose Test (FPG) or an Oral Glucose Tolerance Test (OGTT). Either test can be used to diagnose pre-diabetes or diabetes.

Instead of the person having to go to the care provider, the person may perform the test at home. For example, the person may put a blood sample on a device that is capable of conducting a glucose test.

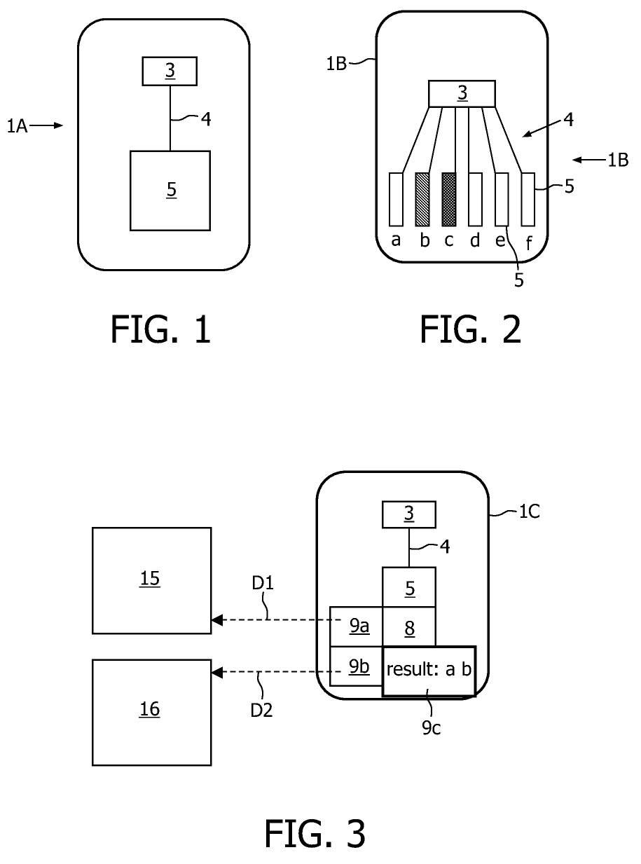

FIGS. 1-3 show various embodiments of assay devices 1 which can be used to perform assays. Each of the assay devices 1 can be configured to assay one or more samples and to provide at least one assay result, for example a result that is based on the assaying of the one or more samples, or, in case the assaying failed: a result which comprises a report stating why the assaying failed.

For example, the devices 1 can be used to assay individual users of an assay system, for screening purposes. Individuals, to be tested by the devices 1, can be human individuals. However, alternatively, the devices can be configured to assay animals or vegetation.

Besides, assays to be conducted with the devices 1 can involve screening of certain other areas or locations, for example screening environments (air, water, soil, etc.) for contamination, for certain substances and/or for organisms.

Each of the assay devices 1, shown in FIGS. 1-3, can be configured to detect various types of analytes. For example, analytes to be determined can include glucose, pregnancy-related analytes, cholesterol, drugs, biotoxins, diseases, cardiac markers, chemicals, hormones, proteins, and/or other analytes. Other analytes can include certain substances, toxic matter, environmental contamination and/or different analytes.

The assay devices 1 can each be configured in various ways to conduct an assay on a sample, as will be clear to the skilled person. For example, the assay device 1 can be provided with one or more suitable enzymes, antibodies, binders or binding agents, a labeling substance, and/or microorganisms, which can be responsive to a specific analyte to be searched for. An analyte and/or analyte-dependent modifications can be detected, for example, optically, electrochemically, by electrical resistance measurement, and/or in a different way, by the assay device 1.

Each of the assay devices 1 can be configured to assay various types of samples, for example samples of blood, bodily fluid, saliva, urine, plasma, serum and/or other sample types, as will be clear to the skilled person.

Besides, advantageously, each of the assay devices 1 is portable, lightweight, and compact, for example having a credit card format configuration. For example, each of the assay devices 1 is configured so as to be capable of being sent to users in a simple envelope or package, or by or as part of a letter, by regular mail. Besides, different assay devices 1 can be sent by different means. Also, for example, each of the assay devices 1 can be disposable. The assay device 1 can be configured for transmitting assay data or information to a remote receiving system (see below). Examples of such assay devices are known to the skilled person, see for example international application WO 95/06240 (Allen) and the above-mentioned WO 95/33996.

Besides, each assay device 1 can comprise a carrier or substrate, and can be provided with at least one sample receiving area 3, to receive a sample, and with at least one detection area 5 for testing a sample received at the receiving area 3 during use. For example, an assay device 1 can comprise one sample-receiving area and one detection area. Alternatively, an assay device 1 can comprise one sample-receiving area and several respective detection areas, for example to assay for different analytes. Besides, an assay device 1 can comprise several sample-receiving areas and several respective detection areas, to test several samples. For example, sample-receiving areas 3 and detection areas 5 can be integrated with each other, or be spaced apart from each other. In the latter case, for example, sample conductors 4 can be provided, for example capillary channels, to conduct one or more samples, or parts thereof, from one or more receiving areas 3 to one or more detection areas 5, for example by capillary action, gravity, or in a different manner. Besides, for example, the assay device can be manipulated, for example via folding or bending, to bring a sample receiving area 3 into contact with a detection area 5. Each assay device 1 can also be configured in a different manner.

Also, each of the assay devices 1 can be configured to provide at least one assay result based on the assaying of the one or more samples. In a simple and relatively inexpensive embodiment, depicted in FIG. 1, the assay device 1A is provided with one detection area 5, which is configured to change color in case a sample contains a specific analyte, to be searched for. If a sample, applied to the respective receiving area, does not contain that analyte, the respective detection area will not change its color. In that case, during use, a respective rest result can simply be the color of the detection area 3.

FIG. 2 depicts a different, for example more complex and/or more sturdy or durable, assay device 1B, which is configured to generate or indicate a code, depending on the presence or absence of the analyte to be searched for. A similar device as such is described in WO 95/33996. For example, the code can comprise an alphanumerical code and/or different symbols and/or characters, which code provides an indication of the test result. As an example, in FIG. 2, various detection areas 5 are provided to test a sample received at a receiving area 3. The detection areas 5 are subsequently marked a, b, c, d, e, f, for example by pre-printed respective markers, to indicate a test result. For example, in FIG. 2, a test result “bc” has been generated by the assay device 1B, resulting from testing a certain sample. For example, in a non-limiting embodiment, the second assay device 1B can be more durable than a first type assay device 1A due to the application of stronger materials, or by using a more durable device packaging and/or due to another reason.

FIG. 3 depicts a more accurate, more complex assay device 1C, which comprises a microelectronic processor 8 to control and process the assaying of a sample. In this case, an electronic test result indicator 9 is provided, controllable by the processor 8 to store and/or indicate a test result. As an example, the processor 8 and test result indicator 9 can be integrated with each other, or be separate components. Also, for example, the embodiment of FIG. 3 can comprise a so-called lab-on-a-chip system, and, for example, the processor 8 can comprise a lab-on-chip processor which can at least partly include a mentioned detection area 5.

In the embodiment of FIG. 3, the test result indicator 9 can be configured in many different ways. In an embodiment, the test result indicator 9 comprises a test result transmitter 9b configured to transmit a test result to an external test result receiver 16. Data transmission between the transmitter 9b and receiver 16 (which data transmission is schematically indicated by a dashed line D2) can, for example, take place via suitable wiring and/or wirelessly, for example using electric, electromagnetic and/or optical signals, a network interface or digital output, or differently.

Also, the test result indicator 9 can be provided, for example, by a memory 9a which can store a test result, and which can be read by an external memory-reading device 15 for obtaining the test result from the assay device 1C. For example, data transmission between the memory 9a and reading device 15 (schematically indicated by a dashed line D1) can take place via suitable wiring and/or wirelessly, for example using electric, electromagnetic and/or optical signals, via a mentioned test result transmitter 9b, or differently.

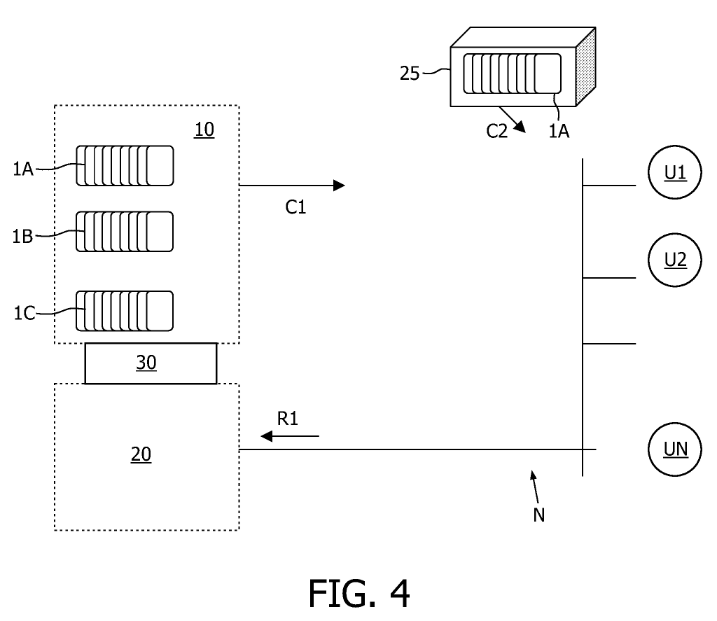

A mentioned external test result receiver 16 and memory reader 15 can be configured in various ways, and can include a dedicated docking station for docking the assay device 1C, a computer, a personal digital assistant (PDA), a mobile phone, and/or can be part of a remote receiving system 20 (see FIG. 4), and/or can be configured differently. For example, in an embodiment, the external test result receiver 16 and memory reader 15 can be integrated with each other.

Besides, for example, in the embodiment of FIG. 3, the test result indicator can comprise an audio and/or visual information generator 9c, for example a display and/or a loudspeaker, to generate audio and/or visual information comprising the test result to be perceived by a user. As an example, in FIG. 3, the indicator is a visual information generator 9c, which displays “result: a b” as a test result. Components of the assay device 1C of FIG. 3 can be powered in various ways, for example by a solar cell, a battery, by charging, by inductance, by self-powering or capillary action, by a storage capacitor, by power storage via motion and/or a winding mechanism, or differently.

In a further embodiment, the assay device is configured to monitor assay context, and preferably to provide the assay data or information with results of such monitoring. For example, the assay device 1 can be provided with one or more assay context sensors to detect temperature, humidity, contamination and/or other assay context factors. As an example, in the FIG. 3 embodiment, one or more such sensors can be integrated in the processor 8, or can be connected thereto in a suitable manner.

Besides, in each of the embodiments of FIGS. 1-3, the assay device 1 can be constructed substantially in one part, wherein the various components are undetachably connected to each other, or can comprise one or more components that are detachable or separable from a remaining device part (in the latter case, for example, similar to the device described in European patent EP0972196B1). For example, in an embodiment, a test result storage part, for example a mentioned memory 9a and/or processor 8 and/or transmitter 9b and/or indicator 9c, might be separable from a respective sample-receiving area 3 of the device 1C. Also, as an option, an assay device 1 can be provided with a test ready indicator, for example via a mentioned test result indicator 9 or otherwise, to indicate when an assay of a sample is completed.

In an embodiment of the invention, the assay device 1 provides assay data or information, the assay data or information relating to, being based on and/or comprising one or more assay results of the assay devices 1. For example, such assay data or information can inform a user of the device 1 directly whether or not a certain analyte has been detected by the device 1. Advantageously, however, the data or information provided by the device 1 is such that it does not give any direct indication to the user whether the analyte has been detected by the device 1. In case an assay fails, the assay data or information can also include information about why the assaying failed, for example failure due to user errors, device errors, passing a device expiration date and other failure types.

In each case, for example, the user of an assay device 1 can send a respective test result (data, information) to a central receiving system 20 (see FIG. 4 and below). Transmission of the test result to the central receiving system 20 can comprise, for example, electronic transmission, transmission via a computer and/or telephone network N, transmission via a communication connection between a user communication terminal and a communication terminal of the receiving system 20, transmission via regular mail and or transmission via locally available test result collection facilities, depending, for example, on the configuration of the respective assay device 1. Also, for example, the user can send (i.e. transmit) the whole assay device 1 or only part thereof, containing the assay data or information, to the receiving system 20.

FIG. 4 depicts an assay system according to an embodiment of the invention. The system can be configured to automatically adapt to a user situation and user motivation, for providing an improved screening method. The assay system comprises a relatively large number of first assay devices 1A, a number of second assay devices 1B, as well as, for example, a number of third assay devices 1C. Each second assay device 1B is configured differently with respect to a configuration of the first assay devices 1A. Also, each third assay device 1C is configured differently with respect to the configuration of the first assay devices 1A, and differently with respect to the configuration of the second assay devices 1B. In a non-limiting example, each first assay device 1A can be similar to the device shown in FIG. 1, each second assay device 1B can be similar to the device shown in FIG. 2, and each third assay device 1C can be similar to the device shown in FIG. 3. As follows from the above, each of the assay devices 1 is configured to assay one or more samples, and to provide at least one assay result, which can be based on the assaying of the one or more samples and which preferably also can contain information regarding assay failure.

The assay system can be provided with a distribution system 10, which is configured to distribute at least one of the second assay devices 1B to a user of the at least first assay device 1A. Preferably, the distribution system 10 is also configured to distribute at least one third assay device 1C to a user of the at least first and/or second assay device. Also, the distribution system 10 can be configured to distribute first assay devices 1A to users. In FIG. 4, distribution of the assay devices 1 is indicated by arrows C1, C2. Users of the system are schematically indicated in FIG. 4 by U1, U2 . . . , UN. The at least one distribution system 10 may comprise, or be coupled to, at least one postal mailing system for distributing the assay devices 1.

In an embodiment, the at least one distribution system 10 can comprise one or more distributors 25 configured to hold a plurality of first assay devices 1A, the distributors 25 being located in or near one of the following: a shopping area, an entertainment area, a public transport area, a hospital, and/or at other locations which might be accessible to (potential) users U. In that way, assay devices 1A can be provided to users in a simple manner, for example without transmitting the devices 1A via mail.

Besides, in an advantageous embodiment, first assay devices 1A can be distributed to users U in combination with products, for example together with food products or consumer products, the products for example being provided with the assay devices 1A. Certain products which might lead to certain health risks, such as products containing high cholesterol and/or high carbohydrate levels, can be sold together with assay devices, configured to screen for such health risks.

In a further embodiment, the system can be configured to send a sequence of assay devices 1A, 1B, 1C to the same user, particularly by sending the assay devices 1A, 1B, 1C successively in time. Preferably, here, the type of a specific assessment device 1 in the sequence depends on previously received results (for example results of the assaying and contextual information about the assaying), of a previously distributed assay device 1, and/or previous knowledge about the user. In an embodiment, different devices 1 can by sent by different mailing means. For example, in case it is determined that transportation of a device 1 by regular mail has disturbed the correct assaying, a second device 1 can be sent in a different manner. For example, a first assaying device might have been frozen in case it was sent by airmail, thus damaging active reagents of the first device, so that a second device is sent by ground/sea-mail, or by airmail in an improved thermally isolating and/or warmed package.

The assay system can also be provided with a mentioned receiving system 20, which is configured to receive assay data or information, the assay data or information relating to, being based on and/or comprising one or more assay results of the assay devices 1, and comprising assay failure data in case of a certain failure or certain failures during the use of the device 1. The receiving of assay data is indicated by an arrow R1. In FIG. 4, both the distribution system 10 and receiving system 20 are schematically indicated by boxes in dashed lines. The assay system 10, 20 can be provided with one or more processing units 30 to control distribution of the assay devices 1 and to control the receiving of assay data, respectively (only one such processing unit 4 is schematically shown in FIG. 4).

Each processing unit 30 can be configured in various ways. For example, each processing unit can comprise one or more processors, computers, memories, timers, micro-electronics, and/or other suitable control unit means, as will be clear to the skilled person. Also, each processing unit can be provided by suitable hardware and/or software. For example, in the latter case, there can be provided a computer program comprising computer-readable instructions, configured to carry out a respective assay method when run by a computer. There can also be provided an information carrier comprising such a computer program.

In an embodiment, during use, the distribution system 10 can send relatively cheap, and relatively inaccurate first assay (or assessment) devices 1A to a set of users, for example via mail. Users U1, U2, . . . , UN may receive the mail, and may be requested (for example via a manual or instructions, provided with the assay device 1A) to perform a respective measurement using the first assay devices 1A, and to return respective assay data to the receiving system 20. For example, the used assay devices 1A or parts thereof can be returned to the receiving system 20 by mail in order to to be processed by the receiving system 20 (and a respective processing unit 30) to obtain and evaluate the respective assay results. Alternatively, users U can transmit assay data via a suitable communication system or network N, for example via a telephone system, computer system, the Internet, and/or other suitable communication means. For example, the receiving system 20 can be provided with a computerized call-receiving system, and/or voice response system, configured to receive calls from the users in order to receive the assay data from the users U, communicate with the users U and/or return test result-related information back to the users U.

This method has important advantages over supervised screening: no medical personnel is required, and the users do not need to travel to a medical processing facility. For example, in an embodiment, the system and method can be used to determine whether or not a person has pre-diabetes or diabetes. Preferably, here, results of the assay are not directly revealed to the user, while the system can still keep users motivated to send in assay results.

In the present embodiment, the system 10 can also distribute second and third assay devices 1B, 1C to the users U. In an embodiment, a difference between the first and second assay device 1A, 1B can be provided in at least one or more of the following device characteristics: assay accuracy, assay sensitivity, robustness, size, shape, weight, analyte, device packaging, functionality, visual feedback to a user, audio feedback to a user, device cost, monitored assay context, assaying procedure, device handling, and rugged or thermally more resistant chemicals. Also, for example, the third assay device 1C can have a higher assay sensitivity than both the first and the second assay device 1A, 1B, the third assay device being more expensive than the first assay device 1A. For example, if results of the first test, using a first device 1, are in a certain range (for example 5.0-6.0), a second assay device 1 can be sent with a higher sensitivity in that range (for example a higher sensitivity in the 4.0-7.0 range). As another example, if results of the first test are >6.0, but the received test context indicates potential contamination, a differently packaged device can be sent with visual feedback to the user (see also below).

In a further embodiment, the distribution system 10 and receiving system 20 can be configured to cooperate in order to distribute at least one second or third assay device 1B, 1C to a user of a first assay device 1A, depending on received assay data or information regarding that first assay device 1A. Such cooperation can be performed, for example, via the mentioned processing unit 30 or a plurality of such processing units 30.

The receiving system 20 can be configured in various ways, as will be appreciated by the skilled person. For example, the receiving system 20 can at least be configured to determine, using received assay data or information, whether a respective assay result of an assay device 1A is a negative or positive assay result, and/or whether the result is inconclusive, and/or whether the assaying has failed and optionally a/the reason(s) why the assaying has failed. Then, in a further elaboration, the distribution system 10 and receiving system 20 can be configured to cooperate (for example via the one or more processing unit(s) 30) in order to distribute at least one further assay device 1B, 1C to a user U of a first assay device 1A, in case the receiving system 20 has determined that a respective assay result of a first assay device 1A is a positive assay result, and/or an inconclusive result. For example, a more accurate third assay device 1C can be sent to the user, who provided a positive or inconclusive test result using a first assay device 1A, to confirm the positive test result or to redo the assay, respectively, with higher accuracy.

Also, in an advantageous embodiment, the receiving system 20 can at least be configured to determine, using received assay data or information, at least one type of deviation concerning received assay data or information with respect to threshold data or information, estimated data or information, and/or expected data or information. In that case, in a further elaboration, the distribution system 10 and receiving system 20 can be configured to cooperate in order to distribute at least one further assay device 1A, 1B, 1C to a user of an assay device (for example a first, second or third assay device 1A, 1B, 1C) in case the receiving system 20 has determined that at least one type of deviation concerning received assay data or information is determined. As a non-limiting example, the deviation can include at least one of the following:
assay data or information is not received within a predetermined time period;
assay data or information received is damaged or at least partly illegible; and
assay data or information received is unreliable. Here, ‘unreliable’ can be defined in various ways, depending on the type of assaying performed. For example, the unreliability can involve assay data information including several data parts that contradict each other, or assay data or information that is improbable or impossible (for example data which would indicate that the temperature during the assay was far below or above an expected or usual assaying temperature).

Thus, a user U can redo the assay, for example, to correct errors or damage relating to a previous assay. Also, a user U might not have received a previously sent assay device, for example due to a mail irregularity, which can be automatically corrected by the sending of a further assay device to the user after the predetermined time period.

For example, in an embodiment, the receiving system 20 can be configured to receive at least parts of distributed assay devices 1, and to perform at least one of the following: detect damage and/or malfunction of received assay devices or of parts thereof, read data or information from received assay devices or parts thereof, recycle received assay devices 1 or parts thereof. For example, to detect damage and/or malfunction of received assay devices or of parts thereof, the receiving system can be provided with suitable sensors and/or detectors, as will be clear to the skilled person. Also, the receiving system 20 can be provided with one or more mentioned test result receivers 16 and/or memory readers 15 to receive assay data and/or to read optional assay memories 9a, if available. The receiving system 20 can be configured, for example, to detect a color and/or optically detectable test result indicators of a received assay device or part thereof.

Besides, in a further embodiment, the assay system 10, 20 can be configured to detect, record and/or memorize a time, and/or location, of distribution of assay devices, and to detect, record and/or memorize a time, and/or location, of reception of assay data relating to assay results of distributed assay devices. Also, for example, a controller unit 30 of the system can be configured to remind a user of a distributed assay device 1 to return assay data a predetermined time period after distribution of the assay device 1 to the user. Such a reminder can be a letter, an email, a telephone call, transmitted to the user via a suitable communication network, and/or a different type of reminder.

In an embodiment, the distribution system 10 can be configured to randomly select users U by randomly selecting user names and/or user addresses from at least one database containing such names and/or addresses, and can be configured to automatically notify selected users to fetch or receive a first assay device 1A, and/or to distribute first assay devices 1A to such randomly selected users. For example, in this way, part of a population can be randomly selected to be screened for one or more health risks or diseases.

Also, in an embodiment, the distribution system 10 can be configured to select users on the basis of one or more medical selection criteria, and can be configured to automatically notify selected users to fetch or receive a first assay device and/or to distribute first assay devices to selected users. For example, the mentioned medical selection criteria can include: gender, age, profession, a known level of participation in sports or exercise, known health disorders of the user, and other criteria. For example, the system 10 can automatically send assay devices 1 to users U, who are known to be overweight and/or who are known not to participate in sport activities, for example to assay the users for (pre-)diabetes and/or cholesterol.

Besides, the distribution system 10 can be configured to select all users by selecting all user names and/or user addresses from at least one database containing such names and/or addresses. In that case, the system can be configured to automatically notify all selected users to fetch or receive first assay devices, and/or to distribute first assay devices to all these users.

For example, the assay system 10, 20 can be part of an insurance management system, or can be operatively connected thereto, wherein all, or a number of, insured individuals are to be assayed for certain health risks. Besides, all who apply to be insured at a respective insurance company can be automatically screened for one or more health risks, using the present system and method, in combination with an insurance management system.

In an embodiment, at least one of the assay devices 1A can include, or can be configured to provide, a lottery ticket. Such an assay device can encourage a user U to perform the assay in a simple and effective manner. For example, in a further elaboration, the system can be configured such that a user can only find out whether the assay lottery ticket is a prize-winning ticket, if the user performs the assay and sends resulting assay data to the receiving system 20. As an example, the assay device can be provided with a respective lottery number, which can be sent to the receiving system 20 together with the assay data, and the receiving system 20 can be configured to notify the user whether his/her lottery number is a prize-winning number.

Besides, in an embodiment, the assay system can be configured to provide a user of a first assay device 1A, in case the user has used the assay device 1A to assay one or more samples, with at least one of the following: a gift, a price, a lottery ticket, a fee reduction, a reduction on an insurance fee. For example, the at least one first assay device 1A can be configured to provide an assay result, which result also determines the gift, price, a lottery number of the lottery ticket, fee reduction, or the reduction on the insurance fee. Referring to FIGS. 2 and 3, the depicted assay result “a b” can be a respective lottery code, or an indication of the level of a price or a fee reduction to be received.

The system depicted in FIG. 4 can be used in an assay method, wherein at least one first assay device 1A is distributed to a user. The first assay device 1A is used by the user to assay one or more samples and to provide at least one assay result based on the assaying of the one or more samples. At least one second and/or third assay device 1B, 1C is provided as well, which are/is configured differently with respect to a configuration of the first assay device 1A. The at least one second and/or third assay device 1B, 1C can also be distributed to the user of the at least first assay device 1A. As follows from the above, for example, the distribution of the second and/or third assay device 1B, 1C can be dependent on the assay result provided by the first assay device 1A. Also, assay results provided by the first assay devices can be transmitted to the receiving system 20, which processes received assay results to determine whether or not a second and/or third assay device 1B, 1C is to be distributed to respective users of respective first assay devices 1A. For example, as follows from the above, in the assay method, at least a second and/or third assay device can be distributed to a user of a first assay device, in case a respective assay result of a first assay device is one of: a positive assay result, an inconclusive result, an unreliable result and an illegible result, or, for example, a respective assay result comprises information that the assaying has failed. Besides, at least a second and/or third assay device can be distributed to a user of a first assay device, in case the receiving system 20 does not receive respective assay data or information, resulting from that first assay device, within a predetermined time period.

Advantageously, there can be provided a method to manage an insurance system, comprising utilizing the described assay system 10, 20 and/or assay method to assay users of the insurance system. For example, users can be encouraged to use the assay devices, in case the insurance system provides a reduction on an insurance fee to such users.

As follows from the above, first, instead of using a single type of assessment device, a screening method can employ a set of different types of screening devices 1A, 1B, 1C. Furthermore, an assay system, for example a processing facility or distribution system 10, can send a sequence of assessment devices 1 to a single user U, where the type of a specific assessment device in the sequence depends on the previously received results and previous knowledge about the user.

In a non-limiting example, the assay system 10, 20 can have three types of assessment devices: a low-cost device 1A, a higher-cost device 1B that is less easily damaged, and a higher-cost device 1C that is more sensitive in a certain measurement range. If the first device comes back broken, the distribution system 10 sends a second device 1B, that is less easily damaged, to the user. If the first device 1A is returned to the receiving system 20, but the first device 1A gives unreliable sensor data, the distribution system can send a more sensitive device 1C to the respective user. Other examples of variations in assay devices are, for example: improved or different user guidance techniques, like not using audio-feedback if it is known that the intended user U has impaired hearing, improved functional capabilities like longer battery-life, larger memory, or more sample application areas or wells.

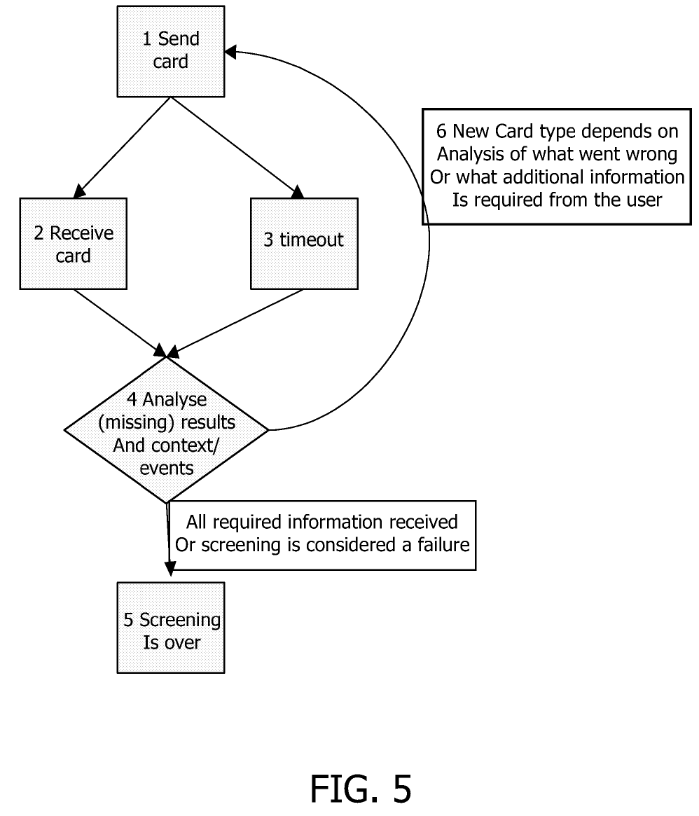

FIG. 5 shows a flowchart of an example of an assay method, which can be operated by the assay system 10, 20, 30. In FIG. 5, in a step 1, an assay card 1A of a specified type is sent to a specific user U. If this is a first mailing, the card type depends on the intended screening procedure and on prior knowledge about the user U. Next, the card 1A is returned and received by the processing facility or receiving system (step 2 in FIG. 5), or, alternatively, the card 1A is not returned and eventually the assay system expects that the card 1A is somehow lost (step 3 in FIG. 5). Both cases are followed by an analysis of the (missing) results and an analysis of the context in which the results were obtained. For example, two outcomes of this analysis can be are possible:
1. The first outcome is that the assay system 10, 20, 30 decides to send an additional card to the user to obtain more and better quality information (step 6 in FIG. 5). This next card can be of a different type that is more suitable to obtain the right test result.
2. Alternatively, a second outcome can be that the assay system 10, 20, 30 decides that no more cards are sent, and the screening is over (step 5 in FIG. 5). Either sufficient information has been received or the screening is considered a failure.

Each of these steps (1-6 in FIG. 5) can be combined with other interactions with the user U, for example a phone reminder or e-mail reminder to send in the card 1A, in case a timeout or expiration is drawing near, or a phone call or e-mail to the user to explain/walk through the assessment procedure or, before completing step 5, inviting the user U to a medical center for a supervised assessment, if the received card results/context show that a supervised assessment is beneficial or the user indicated on the card that he prefers a supervised assessment.

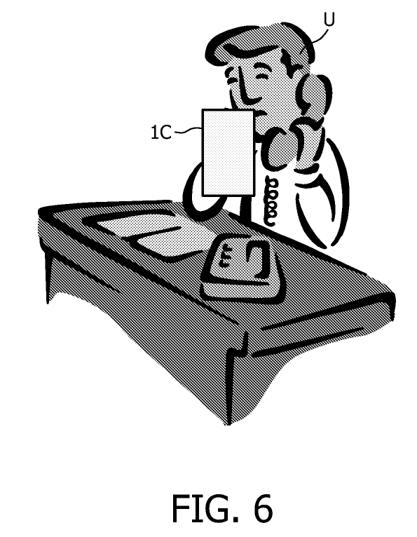

As a further example, users U can simply be motivated by receiving instant feedback on the test results, while it is thus ensured that the test results reach the receiving system 20, as depicted in FIG. 6. To that aim, users U that are expected or known to be motivated by receiving instant feedback are sent a third type of assessment device (or assay device) 1C, that can be extended with the following functionality. The third assessment device 1C can contain an output means like a display (visible output), speaker (audible output) or a network interface (digital output), as follows from the above regarding the FIG. 3 embodiment. The third type of assessment device 1C can be configured to generate an output sequence that encodes a test result and, optionally, test-context and timing information. Either the user U can return the assessment device 1C by mail, or the user can transmit the output sequence to the receiving system 20, as mentioned above. For example, one way of transmitting the output sequence is to use a phone system. The user U phones a dedicated number, and either enters a number sequence shown by the assessment device 1C, or the assessment device 1C generates an audio sequence and the user U holds the assessment device 1C close to the phone. FIG. 6 shows a possible set-up, where the user listens to the phone and holds the card close to a microphone of the phone. The receiving system 20 can either return user guidance by phone, if the sequence is incorrect (in which case the user needs to re-send parts of the sequence, for example by pressing a “replay” button on the card), or the receiving system 20 can decode the sequence, evaluate the test results in real-time, and give the user feedback by phone immediately. This makes it more rewarding for the user U to send in the results, and since the initial output sequence is encoded, the user U is not demotivated from returning the results by the fact that he knows the test result before transmitting it. One of the advantages of this method is that it allows direct interaction about the test result with the user U. However, having human operators performing this interaction can be costly, therefore it is beneficial to provide the receiving system 20 with an automated voice response system instead. However, the results of a test can by quite sensitive and potentially psychologically damaging to a user. It might be that the initial test signals that the user U might be developing, or has developed, a serious health condition. In this case, communicating this result by means of an automated voice response system may aggravate the mental condition of the user. Therefore, the automated response system first analyses the test results, and depending on the outcome and on previous knowledge about the user (like preferences/type of contract), the system either gives an automated response in case the screening detected no issues, or routes the call to a human operator, who is better equipped to handle any questions that a user might have after health issues have been discovered that may possibly have caused him to be in an emotional state. A human operator can provide the user with personal support for dealing with the result and looking towards the future.

A basic idea behind the present invention is the inventive notion that the yield of screening large numbers of individuals, or test sites, can be considerably improved using simple means. Particularly, to this aim, the assay system can provide several assay devices to a single user, if desired, for example in a certain sequence. The system and method can provide relatively accurate screening results using, on average, relatively cheap assaying means, particularly in case very large numbers (for example: many thousands) of individuals are to be screened. Also, users of the system can be motivated in a simple manner to send in assay data or information to provide the assay system with the test results. One application of the invention is to determine whether or not a patient has pre-diabetes or diabetes. However, as an example, testing for other diseases using body fluid samples may also benefit from this invention.

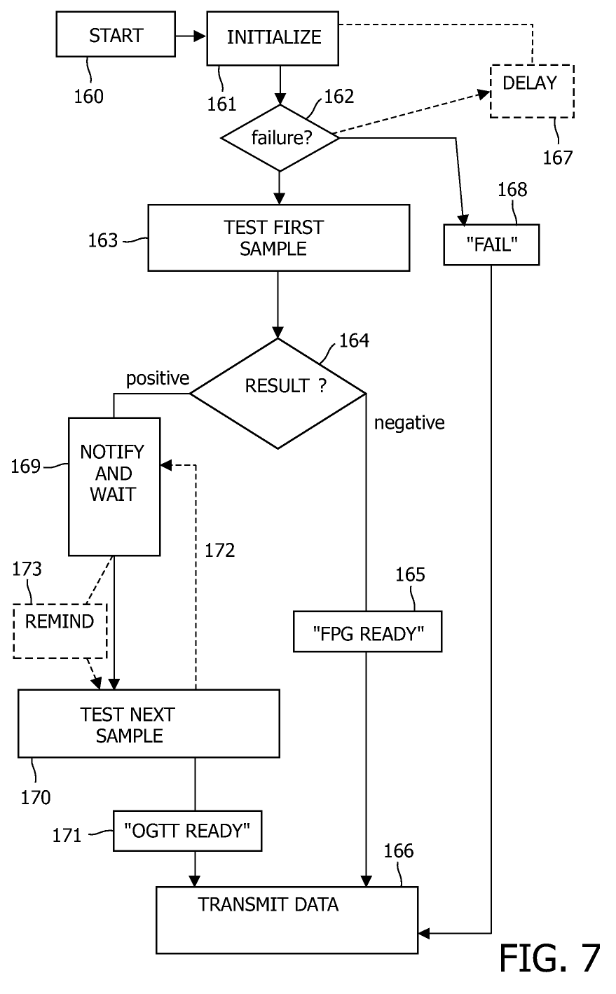

Advantageously, the assay system is, or is also, configured to carry out a relatively precise oral glucose tolerance test (OGTT), utilizing an assay device 1. This will be described in the following, referring to FIG. 7. The embodiment of FIG. 7 can be combined with above-described embodiments, as will be appreciated by the skilled person.

For example, at least one eatable and/or drinkable product can be provided. In that case, the user can be guided to consume the eatable and/or drinkable product before and/or during using the device to assay a sample of the user. In a further embodiment, the eatable and/or drinkable product can contain glucose, and the assay device 1 is configured to test at least one blood sample for glucose. As an example, the product can be a sweet, a wine gum, a glucose-containing beverage, or a different product. Besides, in this case, the disposable assay device 1 can be configured to assay at least two blood samples, for example by being provided with at least two sample-receiving areas 3. For example, a user guidance system can be available to guide the user of the device to test at least a second blood sample after elapse of a predetermined amount of time after testing a first sample. A clock or timer can be provided to measure the elapse of time after the user has applied a first blood sample to a respective application well.

Then, preferably, the assay device 1 can be configured to generate a first test result relating to the assaying of the first sample. Also, the user can be guided to assay at least the second sample, depending on the first test result. For example, a second test can be carried out in case a first test result is “assay inconclusive”, “assay failed”, “device failure” or a similar result. However, preferably, a second test is carried out to turn a FPG test into an OGTT test. As an example, in case the first test result indicates that it is likely that the respective user has (pre-) diabetes, a second glucose test can be performed a predetermined time period after the first test, to provide an OGTT test, to verify the first test result and to provide a much more conclusive OGTT test result. For example, the assay device can be configured to carry out a relatively precise oral glucose tolerance test, if desired. FIG. 7 depicts a flow-chart of a use of such a device.

In FIG. 7, the assaying of a user's blood can be started by the user (step 160), for example by pressing a specific “start test button” S, or giving a command in any other way to the assay device 1.

A subsequent initialization step 162 can involve asking the user questions Q (as in the embodiments of FIGS. 4-5). The outcome of this step 162 can be that the test is not suitable for the user. In that case, the device 1 can indicate “test is not suitable” in a fail-step 168, which test result can be transmitted to a data processing/remote receiving system 20 (step 166).

Alternatively (as indicated by means of broken lines), a result of the initialization step 162 can be that the user has to wait a certain amount of time before he may use the device. Such a delay is indicated by a delay-step 167. Also, during this step 162 it may be determined whether the user is in a fasting state (see above).

On the other hand, in case the initialization phase succeeds, a first user blood sample can be tested by the device 1 in a first blood test (step 163). For example, a first sample-receiving area of the assay device 1 can be used to receive a first blood sample. Optionally, the user can be guided or instructed (for example by a mentioned user guidance system) to consume a mentioned, eatable and/or drinkable product, just before, during or after the application of a user blood sample to an application area of the assay device 1. Preferably, the consumption of the product is at such a time that it does not substantially change the outcome of the first blood test.

In case the first test is “negative” (i.e., pre-diabetes or diabetes is unlikely), a “test ready” indication can be provided (for example “FPG ready”, see step 165). Also, information or data relating to the test result can be transmitted to a processing facility 20 (step 166). For example, in the case that the user was in a fasting state just before taking his first blood sample, the first test result can be an FPG test result.

On the other hand, the device 1, or a user guidance system, may desire that a more accurate OGTT is performed. This can be the case, for example, when it had been found in the initialization step 162 that the user was not in a fasting state. Also, the OGTT test may be desired in case the first test result was positive (i.e., there is a likelihood of pre-diabetes or diabetes).

To perform the OGTT test, the device 1 (or user guidance system) can notify the user to wait a predetermined amount of time (step 169; for example 1 hour, 2 hours or a different period). The user can be instructed to take a second blood sample and test the sample, using a second sample-receiving area of the device 1, immediately after lapse of the waiting period. Preferably, a reminder that the waiting period is about to lapse and/or has just lapsed is provided by a user guidance system (step 173), for example via an alarm of the device 1 or via a call from a remote call center. Then, a second blood sample can be applied and tested (step 170), using the device 1. Thus, an oral glucose tolerance test can be carried out. Optionally, after the testing of a second blood sample, one or more blood samples can be tested after predetermined waiting periods (step 172). Preferably, the device 1 measures the amount of time that has lapsed between the application of the various blood samples to respective application wells, and stores the lapsed time period(s), or stores the times that the samples were applied to the device 1.

After completion of the testing of the at least first and second blood samples, a test ready signal can be provided (for example “OGTT ready”, see step 171), and resulting test information can be transmitted in a suitable manner (step 166).

An advantage of the OGTT test is that it is much more reliable than the FPG test. In this way, for example, the assay device 1 can at least perform, or try to perform, a relatively fast FPG test on a user blood sample. Depending on the outcome of this test, or depending on the user's condition, the test can be changed into the OGTT test. In the latter case, the FPG test can simply be used as part of the OGTT test. In a non-limiting embodiment, an above-mentioned distribution system 10 can be configured to distribute at least one, second assay device, suitable to carry out the OGTT test, to a user who has already used a simpler or less precise assay device.

Although the illustrative embodiments of the present invention have been described in greater detail with reference to the accompanying drawings, it will be understood that the invention is not limited to those embodiments. Various changes or modifications may be effected by one skilled in the art without departing from the scope or the spirit of the invention as defined in the claims.

It is to be understood that in the present application, the term “comprising” does not exclude other elements or steps. Also, each of the terms “a” and “an” does not exclude a plurality. Also, a single processor or other unit may fulfill functions of several means recited in the claims. Any reference sign(s) in the claims shall not be construed as limiting the scope of the claims.

1. Assay system, comprising:
at least one first assay device (1A) that is configured to assay one or more samples, and to provide at least one assay result;
wherein the assay system is characterized by:
at least one distribution system (10) which is configured to distribute at least one second assay device (1B) to a user of a first assay device (1A).

2. Assay system according to claim 1, the difference between the first and second assay device (1A, 1B) being provided in at least one or more of the following device characteristics: assay accuracy, assay sensitivity, robustness, size, shape, weight, analyte, device packaging, functionality, visual feedback to a user, audio feedback to a user, device cost, monitored assay context, assaying procedure, device handling, and rugged or thermally more resistant chemicals.

3. Assay system according to claim 1, comprising at least one receiving system (20) which is configured to receive assay data or information, the assay data or information relating to, being based on and/or comprising one or more assay results of the assay devices (1) and/or comprising information that the assaying has failed.

4. Assay system according to claim 3, the distribution system (10) and receiving system (20) being configured to cooperate in order to distribute at least one second assay device (1B) to a user of a first assay device (1A), depending on received assay data or information.

5. Assay system according to claim 3, the receiving system (20) at least being configured to determine, using received assay data or information, whether a respective assay result of an assay device (1) is a negative or positive assay result, and/or whether the result is inconclusive.

6. Assay system according to claim 5, the distribution system (10) and receiving system (20) being configured to cooperate in order to distribute at least one further assay device (1B, 1C) to a user of a first assay device (1A), in case the receiving system (20) has determined that a respective assay result of a first assay device (1A) is a positive assay result, and/or an inconclusive result.

7. Assay system according to claim 3, the receiving system (20) at least being configured to determine, using received assay data or information, at least one type of deviation concerning received assay data or information with respect to threshold data or information, estimated data or information, and/or expected data or information, wherein the distribution system (10) and receiving system (20) are preferably configured to cooperate in order to distribute at least one further assay device (1A, 1B, 1C) to a user of an assay device (1A, 1B, 1C), in case the receiving system (20) has established that at least one type of deviation concerning received assay data or information is determined.

8. Assay system according to claim 7, the deviation including at least one of the following:
assay data or information is not received within a predetermined time period;
assay data or information received is damaged or at least partly illegible; and
assay data or information received is unreliable.

9. Assay system according to claim 1, being configured to detect, record and/or memorize a time, and/or location, of distribution of assay devices, and to detect, record and/or memorize a time, and/or location, of reception of assay data relating to assay results of distributed assay devices, wherein the system is preferably configured to remind a user of a distributed assay device (1) to return assay data a predetermined time period after distribution of the assay device (1) to the user.

10. Assay system according to claim 1, configured to send a sequence of assay devices (1, 1A, 1B, 1C) to the same user.

11. Assay method, comprising:
distributing at least one first assay device (1A), to be used by a user to assay one or more samples; and
providing at least a second assay device (1B);
distributing the second assay device (1B) to a user of the first assay device (1A).

12. Assay method according to claim 11, wherein the distribution of the second assay device is dependent on an assay result provided by the first assay device.

13. Assay method according to claim 11, wherein assay results provided by the first assay devices are being transmitted to a receiving system (20), which receiving system processes received assay results to determine whether or not a second assay device is to be distributed to respective users of respective first assay devices.

14. Assay method according to claim 11, wherein at least a second assay device is distributed to a user of a first assay device, in case a respective assay result of a first assay device is one of: a positive assay result, an inconclusive result, an unreliable result and an illegible result, and wherein preferably at least a second assay device is distributed to a user of a first assay device, in case the receiving system (20) does not receive respective assay data or information, resulting from that first assay device, within a predetermined time period.

15. Assay method according to claim 11, comprising providing at least a third assay device (1C), the third assay device being configured differently with respect to the configuration of the first assay device (1A), and being configured differently with respect to the configuration of the second assay device (1B), wherein the third assay device (1C) can be distributed to a user of the at least first and/or second assay device (1A).
